(19) United States
(12) Patent Application Publication
(10) Pub. No.: US 2011/0144600 A1
(43) Pub. Date: Jun. 16, 2011

(54) System for receiving oral human discharge

(76) Inventors: Judith Shoemaker, Marlton, NJ (US); David Shoemaker, Marlton, NJ (US)

(21) Appl. No.: 12/653,322

(22) Filed: Dec. 11, 2009

(51) Int. Cl. A61M 1/00

(52) U.S. Cl. 604/322

(57) ABSTRACT

A system for receiving oral human discharge which includes a pail, container, or like receptacle having an open space for the receipt of such discharge, e.g. vomit, phlegm, etc. The receptacle is configured to be positioned within an opening in the upper section of a stand. An intermediate section extends between the upper section and a lower section. The lower section is designed to be removeably inserted between the mattress and frame platform or box spring of a bed or the cushions of a couch. The stand and, hence, the receptacle are thus positioned directly adjacent to the sick child, providing ready access for immediate use. Two receptacles are provided in stocked relation, so that when one is being cleaned, the other remains within the opening in the stand, ready for use.

### BACKGROUND OF THE INVENTION

Sick children are sometimes very difficult to care for, and this is especially the case where one of the conditions of the child's illness involves vomiting or spitting up. The particular illness presents stress enough, without both parent and child/patient having to worry about the physical consequences, including clean-up, of their child's need to throw up. Of course, the child is obviously upset. And while the parent is most concerned with the child's illness, when vomiting is an issue, especially when it is unexpected or intermittent, there is an added degree of anxiety.

This problem has long been addressed solely by the use of buckets or waste receptacle containers which are merely just kept by the bedside of the sick child. However, such containers often cannot be readily located when needed immediately, are inaccessible, or, once they are used, are not easily available for immediate reuse. There is currently no system for receiving oral human discharge, e.g. vomit, phlegm, spit up, which addresses these problems.

### SUMMARY OF THE INVENTION

It is thus the object of the present invention to address the problems associated with attending to vomiting and spitting up resulting from illness, especially the illness of children.

The system for receiving oral human discharge of the present invention is designed to relieve the anxiety of vomiting for both children and their parents, by providing a container or receptacle which is placed close to a bed or couch, thus making the experience of being sick less stressful. The system relieves the anxiety of sick children who ordinarily can not make it to the bathroom quickly or who are concerned about locating a container into which to throw up while in bed during the night or while napping on the couch or like furniture. In addition, the container or receptacle of the present invention can be designed to give children something pleasant to look at, even when they are not feeling well.

The system of the herein invention also reduces parental stress in that it helps to eliminate the potential mess caused when the child is sick while on a bed or couch. Cleanup, once the child vomits or spits up into the container of the present system, is sanitary, quick, and simple. Use of the system also avoids the panic which results in attempting to timely transport the child to the bathroom. Of course since the child's anxiety is eliminated, parents feel better as well.

These and other objects are accomplished by the present invention, a system for receiving oral human discharge which comprises a pail, container, or like receptacle having an open space for the receipt of such discharge, e.g. vomit, phlegm, etc. The receptacle is configured to be positioned within an opening in the upper section of a stand. An intermediate section extends between the upper section and a lower section. The lower section is designed to be removeably inserted between the mattress and frame platform or box spring of a bed or the cushions of a couch. The stand and, hence, the receptacle are thus positioned directly adjacent to the sick child, providing ready access for immediate use. Two receptacles are provided in stocked relation, so that when one is being cleaned, the other remains within the opening in the stand, ready for use.

The novel features which are considered as characteristic of the invention are set forth in particular in the appended claims. The invention, itself, however, both as to its design, construction and use, together with additional features and advantages thereof, are best understood upon review of the following detailed description with reference to the accompanying drawings.

### BRIEF DESCRIPTION OF THE DRAWINGS

FIG. 1 is a view of the system of the present invention in use.

FIG. 2 is an exploded view of the components of the system of the present invention.

FIG. 3 is a view of the receptacle of the system of the present invention.

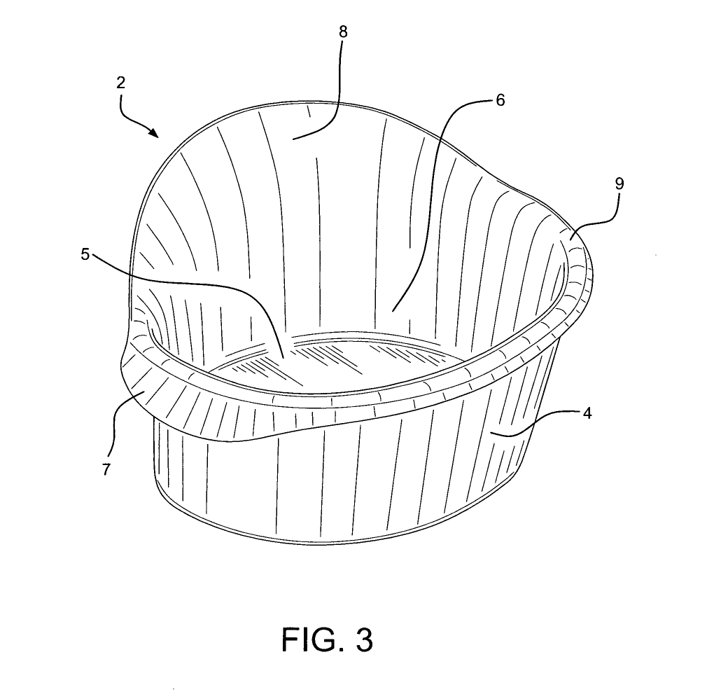

FIG. 4 is a front view of the receptacle of the system of the present invention.

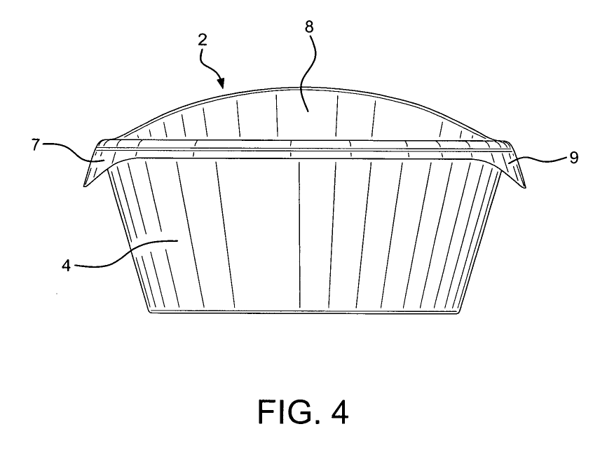

### DETAILED DESCRIPTION OF THE INVENTION

System 1 of the present invention comprises vomit receiving receptacles 2 and 3 which can take the form of a pail, bucket, or like container. Receptacle 2 has encircling outer wall 4 and bottom 5. Outer wall 4 encompass open space 6 into which human oral discharge, e.g. vomit, phlegm, etc. is retained. Outer wall extension 8 emanates from a section of outer wall 4 for protecting oral discharge splash or spillover. Handles 7 and 9 extend laterally from outer walls 4.

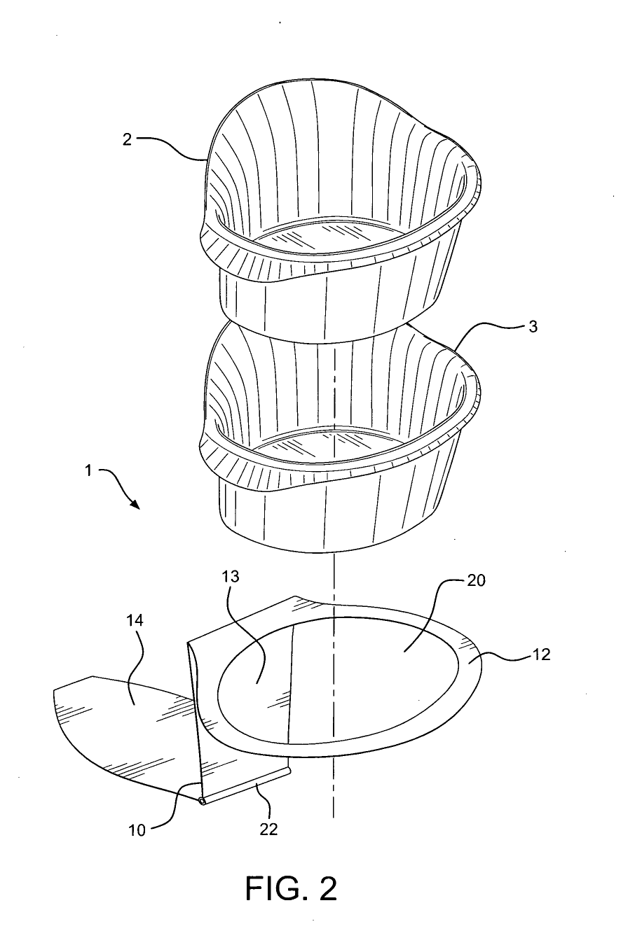

Stand 10 is designed to support stacked receptacles 2 and 3 in spaced relation to a bed, couch, or other user occupied furniture. Stand 10 comprises upper section 12, lower section 14 and intermediate section 13 extending between the upper and lower sections. As best seen. in FIG. 2, upper section 12 and lower section 14 extend perpendicularly, in opposite directions in relation to the intermediate section 13.

Upper section 12 has opening 20, configured to accept and retain receptacles 2 and 3. These receptacles are readily lifted up from opening 20 and removed when necessary, e.g. for cleaning or for transport of system 1.

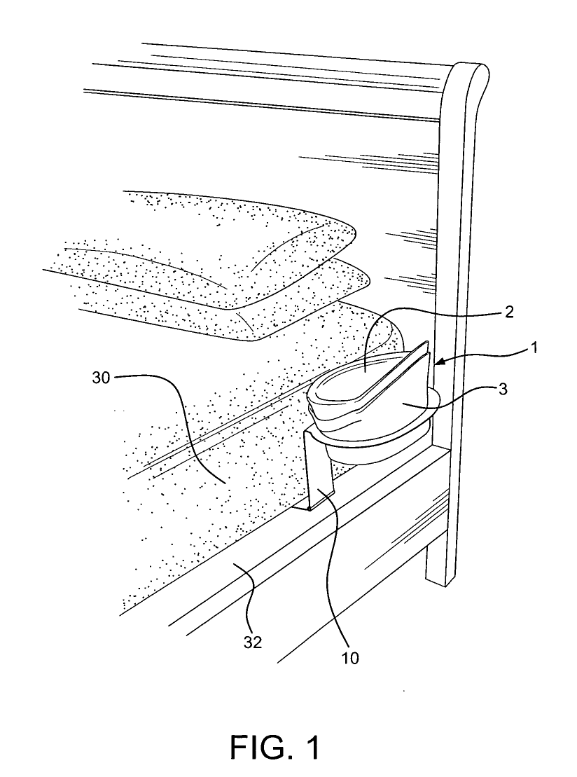

Lower section 14 is formed as a flat, planar member, configured to be slid between user occupied furniture, e.g. the mattress 30 and frame platform or box spring 32 of a bed (FIG. 1), or the cushions of a couch. System 1 can be easily removed by simply sliding lower section 14 out from the bed, couch or like furniture.

Convenient transport system 1 is accomplished by use of hinge member 22, provided between lower section 14 and intermediate section 13. Hinge 22 allows pivoting of the lower section 14 upwards adjacent to intermediate section 13. Hinge 22 can be any standard hinge, lockable at a 90° angle.

In use, lower section 14 of stand 10 is slid between and so is appended from e.g. a bed's frame, platform or box spring 32 and mattress 30, such that upper section 12 extends outward, away from but adjacent to the side of the mattress, as seen in FIG. 1. Receptacles 2 and 3 are placed within opening 20 of upper section 12, making them easily and readily accessible to the sick child in the bed. After oral discharge, e.g. vomit, phlegm, etc., into receptacle 2, that receptacle is removed for discharge disposal and cleaning and receptacle 3 is left in place for use if needed.

Receptacles 2 and 3 can be colored and can comprise different designs specifically for boys or for girls. Words of encouragement can be printed on the inner surfaces of outer walls 4, e.g. “Stay Calm”, “Relax”, “Feel Better Soon”.

While it is anticipated that the system of the present invention is most advantageously used by children, it can certainly be used by adults of all ages, and it has special value to seniors and disabled individuals.

Certain novel features and components of this invention are disclosed in detail in order to make the invention clear in at least one form thereof. However, it is to be clearly understood that the invention as disclosed is not necessarily limited to the exact form and details as disclosed, since it is apparent that various modifications and changes may be made without departing from the spirit of the invention.

1. A system for receiving oral human discharge comprising:
receptacle means for retrieving and collecting oral human discharge, said receptacle means having outer walls encompassing an open space for receiving said discharge; and
stand means for supporting the receptacle means in spaced relation to user occupied furniture, said stand means comprising an upper section having opening means for accepting and removeably holding the receptacle means therein, and lower section means for removeably appending the stand means to the occupied furniture, whereby upon appending the lower section to the furniture, the user has direct access to the receptacle means for purposes of oral discharge and following discharge, the receptacle means is configured to be manually lifted up and removed from the stand means for discharge disposal and cleaning.

2. The system as in claim 1 further comprising an intermediate section between the upper section and the lower section.

3. The system as in claim 2 wherein the intermediate section is perpendicular to both the upper and lower sections.

4. The system as in claim 3 wherein the upper and lower sections extend in opposite directions in relation to the intermediate section.

5. The system as in claim 2 wherein the upper and lower sections extend in opposite directions in relation to the intermediate section.

6. The system as in claim 1 wherein the lower section means is a flat, planar member, configured to be slideable within said user occupied furniture.

7. The system as in claim 1 wherein the receptacle means comprises a solid container.

8. The system as in claim 1 wherein the receptacle means comprises a plurality of stacked containers.

9. The system as in claim 1 wherein the receptacle means further comprises laterally extending handles.

10. The system as in claim 1 wherein the receptacle means comprises extension means emanating from a section of the outer walls for protecting from oral discharge spillover.

11. The system as in claim 1 wherein the lower section means is pivotable in relation to the intermediate section.

12. A system for recovering oral human discharge comprising:
at least one receptacle having outer walls encompassing an open space;
a receptacle stand comprising:
an upper section having opening means to removeably accept and retain at least one receptacle upon insertion of said receptacle therein;
an intermediate section having two ends, the first end extending substantially perpendicularly downward from the upper section; and
a lower section extending substantially perpendicularly outward from the second end of the intermediate section and extending in the opposite direction as the upper section, said lower section comprising a flat planar member being configured to be slid within user occupied furniture.

13. The system as in claim 12 comprising a plurality of stacked receptacles.

14. The system as in claim 12 wherein the receptacle means further comprises laterally extending handles.

15. The system as in claim 12 wherein the receptacle means comprises extension means emanating from a section of the outer walls for protecting from oral discharge spillover.

16. The system as in claim 12 wherein the lower section means is pivotable in relation to the intermediate section.
